(19) United States
(12) Patent Application Publication
(10) Pub. No.: US 2018/0368819 A1
(43) Pub. Date: Dec. 27, 2018

(54) A Smart Bio-Fluids Diagnostic Monitoring System And A Method Of Monitoring Healthiness

(71) Applicant: Gautam Gogineni, Guntur (IN)

(72) Inventor: Gautam Gogineni, Guntur (IN)

(73) Assignee: Shyamala Devi Gogineni, Hyderabad, Telangana (IN)

(21) Appl. No.: 16/062,155

(22) PCT Filed: Dec. 19, 2016
(86) PCT No.: PCT/IB2016/057761

§ 371 Date: Jun. 14, 2018

(30) Foreign Application Priority Data
Dec. 22, 2015 (IN) 5693/CHE/2015

(51) Int. Cl. A61B 10/00; E03D 13/00; A61B 5/145; A61B 5/00

(52) U.S. Cl. CPC A61B 10/0051; E03D 13/005; A61B 5/1171; A61B 5/6887; A61B 5/14507

(57) ABSTRACT

A smart bio-fluids diagnostic monitoring system and a method of monitoring healthiness according to our invention comprise of a semi-portable potty/commode that conserve water having provision for managing waste disposal with built-in self-service health lab and a monitor that connects to the internet to show instant personal health reports. It's matched with a big data platform and personal data control for mass medical research and financial program to benefit 3rd worlds and medical conglomerates. It's a solution with personal, commercial and social implications.

### FIELD OF INVENTION

The present invention generally relates to a Smart Health Monitoring, Analysis and Remediation Tracking system based on Bio-fluid which is a smart bio-fluids diagnostic monitoring system with optimized direct specimen capture and mining with remote configuration and management herein after referred as (SHMART) system. More particularly the invention relates to a system and method of instantly, continuously, accurately and cost-effectively monitoring the healthiness of an individual by bio fluid analysis, without direct involvement of a medical professional, and are deployable on a massive scale, allowing timely reverse-track epidemics during initial breakouts itself.

### BACKGROUND OF THE INVENTION

According to experts, most pathogens, bacteria, & viruses are found internally within the human body. 85% of them are in the guts & digestive canal. So internal bio-fluid monitoring is a far better leading-indicator of wellness, infection and decease compared to superficial monitors that measure factors such as blood-pressure, heartbeats, temperature, and level of exertion from exercise/workouts indicate symptoms whereas the biofluids monitors point to root-causes.

This invention, smart bio-fluids diagnostic monitoring system is designed to achieve Preventive Healthcare on a Scale and Precision never-ever achieved before. It is designed to shift the paradigm of the Healthcare Ecosystem from “Provider driven reactionary remediation of health threats” to “Highly sensitive proactive monitoring of individual health and fully-controlled regulation-compliant data channels with ability to prove economic incentives for a balanced Health-care ecosystem. In order to understand the efficacy of the invention one needs to start by considering the critical-factors of Preventive Healthcare.

It is well established that “Healthiness is best achieved preventively”, rather than dispensing remedies reactively. That applies universally whether it is an individual health—fighting decease, defying aging, or simple health maintenance; OR an entire population—fighting an epidemic, dispensing healthcare broadly or managing costs.

Improving Preventive Healthcare requires “Contiguous Health Monitoring” of “Meaningful Facts”, with ability to do “Early Trend Analysis” and “Timely Follow-up” of the best available remediation option. Examining each of these factors in detail:

Contiguous Health Monitoring

Contiguous Health Monitoring is defined as a system that monitors Meaningful Facts with adequate frequency on a continuous basis for all individuals within a given population over a long-term.

Contiguous Health Monitoring can only be kept-up-with regularly in the long-term if the dependent system is consistent, unobtrusive to lifestyle & livelihood of the individual and conducive to the general dynamics of the locally available healthcare ecosystem.

Scope of Monitoring must have the entire population under consideration. Remote areas, regions with natural & war calamities, impoverished populations, and rapidly contagious decease pose the greatest risk to healthcare safety and cost.

Meaningful Facts

Meaningful Facts are defined as a set of facts that are complete in describing all the causative critical-factors and have the ability to be accurately measured and compared to known tolerances or documented for further analysis.

In other words, Meaningful Facts are a combination of Quality Measurements of the Most Relevant Facts. Meaningfulness of Facts is increased when
a. Measured at the Primary Causative Events rather than Secondary Dependent Events.
b. Measurement in conducted in close Physical and Chronological Proximity to the Causative Event.

Early Trend Analysis

Early Trend Analysis is defined as the ability to scrutinize a collection of Critical Volume of Meaningful Facts to identify if one or more component datum has crossed a known tolerance limit or identifies with a more complex recognized pattern or helps decipher a newly trending pattern that may predict a forthcoming Event of Interest. For the purposes of this disclosure a Pattern is defined as any recognized set of facts that may indicate a future Event of Interest. An Event of Interest may be either a health concern or an indication of remedy. So a Pattern may favorable or unfavorable depending on the context. The goal of Early Trend Analysis is to trigger Timely Follow-up at the first suspicion of the Critical Fact of such match in Pattern.

Analysis of Patterns requires certain critical volume of data in order to contain sufficient repeats of the pattern to infer its existence. The volume of data required varies based on complexity of the pattern and varying sensitivity of the Analytical Process (human, artificial or comparative) that is used.

Compilation of such critical volume of data may expend more time than may be available to handle the individual case-at-hand, especially if type of event or pattern is new or yet to be discovered. Trend Analysis must continue even in such cases, primarily to aid future occurrences but also in anticipation of any corrective action that may be still be available for the case-at-hand based on the new pattern information.

Timely Follow-Up

Timely Follow-Up is defined as triggering any Action(s) that will logically take advantage of the Pattern recognized in the Early Trend Analysis. Such Actions may vary widely based on the context from a simple request to recertify the Pattern and eliminate any False Positives; increasing sensitivity to subsequent Patterns; initiation of known remedies; and documenting for knowledge dissemination and requests to investigate remedies.

Timeliness is key to the efficacy of the Follow-up Action as Early Trends found in the Analysis may develop exponentially over time and impact the nature of remedy drastically. Most events are connected—Many bodily functions & ailments correlate; decease exhibit patterns of manifestation; epidemics are contagious; development of remedies & resources work best when leveraged; neutralization of costs are reduced by mass and distributed business drivers mobilize commercial participation. For that reason, Preventive Healthcare must be proactive throughout the healthcare ecosystem. This requires a system that is well integrated with the Healthcare ecosystem and the related Services and Resources.

SHMART system addresses the limitations of these currently available and State-of-the-Art products by smartly integrating several leading technologies as explained in this disclosure.

### Prior Art and Problem Solved

The systems and services known in the prior art patents and in other literature include traditional diagnostic labs, state-of-the-Art and trending “Smart Watch” technologies with monitoring apps installed, etc. However none of the devices/services found in the market or published research documents have a solution to contiguously and cost-effectively monitor healthiness for all members of a given population. Further they lack the accuracy afforded by a bio fluid analysis, without direct involvement of a medical professional. They are neither deployable on a massive scale especially in rural areas nor allow timely reverse-tracking of epidemics during initial breakouts.

Some of the prior art patents related medical diagnostic method and systems for monitoring the health of individual are mentioned here by way of reference. viz. U.S. Pat. No. 6,170,318; U.S. Pat. No. 7,734,718; U.S. Pat. No. 7,822,454; U.S. Pat. No. 8,668,656; U.S. Pat. No. 8,870,762; U.S. Pat. No. 8,945,910; U.S. Pat. No. 8,962,346; and US20060178841. But these existing systems available at present globally have a lot of limitations. Some of them are enumerated below.

Following are the details of available alternatives and corresponding limitations listed by solution-category:
DIAGNOSIS is currently achieved using disparate units of equipment and service offerings with cost, time and risk implications that force “reactive diagnostics” (i.e. diagnosis only when there is reasonable suspicion of need) rather than allowing regular proactive monitoring of internal diagnostic indicators such as fecal examination.
EPIDEMIC DECEASE TRACKING during pandemic breakouts and natural or other calamity places undue burden on medical teams serving stricken areas to use manual out-of-band channels to capture information that correlates the patient, their whereabouts, symptom and decease, so as to trace down and address the decease breakout situation. It also causes delays when early detection is critical in controlling such breakouts and remedying the decease.
MONITORING is currently achieved using smart-watch type external monitors that instantly and rather-continuously document heartbeats; blood pressure; estimate of calories spent and detail workout affects. Such monitors are external to the body, cannot track infections and decease and hence limiting to what can be monitored superficially.

Use of a Toilet facility regularly is a natural routine followed by all living beings and hence is highly suitable point of monitoring data collection compared to Activity Tracking Devices and other self-help techniques that are becoming omnipresent.

To maintain consistency of monitoring, users need to be proactive, i.e. undertake an activity (i.e. run, walk, and carry the device, provide manual input (type, quantity, and quantity of diet) regularly & routinely. Else the resulting reports will suffer from incompleteness. Completeness of analysis provided by the SHMART system according to our invention works even in cases of passive users which is extremely important in cases of acute or chronic disease management, senior health and end-of-life care.

Current self-help monitors are typically physically connected to individuals and switching between individuals is not generally feasible or needs reconfiguration. Thus requires acquiring and maintaining multiple units. They may also needs cross-product integrations based on application preferences of individuals.
DIET monitoring and calories intake is usually estimated with manual input which is subjective and sporadic. Due to such factors, existing diet monitoring is in most cases discontinued within months if not days.
IDENTIFICATION of healthcare information with patient is governed by several regulatory laws especially in large markets. E.g. US Healthcare laws require compliance with HIPAA regulations mandating strict User Identification control. Similarly US FDA regulations and other standards require pharmaceutical companies to conduct “double-blind” & other compliance concerns cause identification issues.

Current State of the Art Implementation can be summarized as under:

Taking the domain in its entirety, there are currently 2 main arrangements that can be considered as competing solutions. These are:

a) Diagnostic Labs:

These are the traditional diagnostic labs providing medical measurements through doctors and other medical practitioners and are primarily accessed through Health Insurance schemes.

The traditional medical providers and diagnostic labs cater to the health needs of this domain but the focus is biased towards remediating after well-matured patterns emerge and the decease is deeply manifested rather than prevention. This approach has radically increased the cost of remediation. Added to that the towering costs incurred from pharmacological research to dispensing health and geographical reach have limited the efficacy of the system to the point that even in well-developed countries 15% of the economy is attributed to healthcare.

General preventive efforts are limited to intervals of annual doctor check-ups for those covered by health insurance. BioFluid diagnosis is heavily relied upon but is only undertaken if and when an ailment becomes so manifested that it becomes otherwise apparent. That frequency is grossly insufficient for being considered proactive especially for serious fast-developing deceases.

Moreover, deployment in infrastructure deprived remote locations where epidemics hit becomes very expensive and costs precious time, thus reducing the service to a triage catering to the seriously ill and ignoring opportunity for action based on early trend analysis.

b) Exterior Activity Tracking Devices:

There are the new consumer electronic devices those are either wrist-worn or carried in pockets that primarily measure physical activity. These measure superficial physical attributes such as number of steps taken, sleep cycles and other activities and the most sophisticated of which even measure heartbeats. Such tools are considered State-of-the-Art and have become quite popular.

The limitation of these Activity Tracking devices is that they are only measuring superficial attributes only useful for the highly-active individuals and their level of sophistication is limited to consumer levels of interaction. They rely on the individual's knowledge and textual input to document even the basic information of how many calories of food was consumed. This solution does not to cater significant parts of the population who are disabled, sick and bed-ridden, old or infants or illiterate. Thus Activity Tracking Devices is limited to able individuals self-monitoring for physically manifesting decease and are of limited utility towards preventive healthcare.

So in summary there is neither a single system nor a combination that exists to address all the requirements of a Preventive Healthcare system. Here I summarize some of the severely limiting in several aspects of the combination both Diagnostic Lab Provider (DLP) and the Action Tracking Device (ATD) solutions discussed above.
1. Significantly delayed warnings of detection and remediation response times and inability to detect sparse infections.
2. Inability to contiguously operate in the long-term especially in the remotely located populations
3. Obligation to compromise between prohibitively expensive deployments or reduced meaningfulness of measurements of the secondary facts captured.
4. Lack of ability to cater to significant number of member categories of a population. Most needy members are conspicuously omitted including people with low mobility, sick and busybodies.
5. Lack of integration or economic drivers for development of Remediation services

As explained above none of the devices/services found in the market or published research documents have a solution to instantly, continuously and cost-effectively monitor healthiness with the accuracy afforded by a bio fluid analysis, without direct involvement of a medical professional, nor are they deployable on a massive scale or allow timely reverse-tracking of epidemics during initial breakouts. But use of a Toilet facility regularly is a natural routine followed by all living beings and hence is highly suitable point of monitoring data collection compared to all other smart heath monitoring devices and other self-help techniques that are becoming omnipresent.

The present invention addresses the limitations of these currently available and State-of-the-Art products by smartly integrating several leading technologies as disclosed herein.

The documentation and materials yielded from the search for “Smart Toilet’ and “Toilet Diagnostics” were limited. It included concepts and designs regarding intelligent positioning of toilet-seats and some references to environmentally friendly toilet water & waste management and some wishful thinking. No details found on diagnostic tools, processes or Internet-of-Things connections for toilets or biofluids, professional level automated diagnostic analysis or monitoring. Traditional diagnostic labs are available through medical providers however are cost-prohibitive to use for regular monitoring; State-of-the-Art Activity Tracking Devices technologies with health monitoring applications are limited to superficial monitors and also dietary monitoring relied on manual & subjective user input.

SHMART system addresses the limitations of these currently available and State-of-the-Art products by smartly integrating several leading technologies as explained in this disclosure.

Hence we have come out with a state of the art smart bio-fluids diagnostic monitoring systems and method device/paraphernalia with optimized direct specimen capture and mining with remote configuration and management.

Therefore the main objective of the present invention is to have the state of the art smart bio-fluids diagnostic monitoring device which can instantly, continuously, accurately and cost-effectively monitor the healthiness of an individual by bio fluid analysis without direct involvement of a medical professional while also allowing compliance with laws of Health Data Privacy.

### SUMMARY OF THE INVENTION

The invention relates to a smart bio-fluids diagnostic monitoring system and a method of monitoring healthiness.

According to the invention the Smart Health Monitoring, Analysis and Remediation Tracking (SHMART) system based on Bio-fluid comprising of the following. Biometric Identifiers (1) that automatically and seamlessly identify or validate the user of the SHMART system; IOT-Processor (2), a micro-processing computer that is designed for Internet-of-Things; a set of sensors selected (3) from Ambient sensors, Perfunctory sensors, Supply Sensors and Biometric; Macerator (4) to homogenize the consistency of the bio-fluids; Piezo Electro-Mechanic devices (5) to convey tightly-controlled quantities of fluids into the necessary SHMART components as necessary; Specimen Cartridges (6) for holding in-process BioFluids in preparation of diagnostics; Diagnostics Equipment (7) compatible for deploying in the SHMART system; Supply Cartridges (8): required by the diagnostic equipment; User Interface (9) that allows the user to interact with the System; Local database (10) an electronic store that holds the data; Onboard Analytics (11), a set of rules forming the 1st level of intelligence of the system; IOT-Network (12) that helps to communicate with rest of the network; BlockChain (13), a data security mechanism; Bio-Info Wallet (14), a software folder that contains a key to all the diagnostic information associated with a particular user; Cloud Data Base (15); Centralized Analytics (16; and Federated & Distributed Analytics (17); all of which are configured to work with semi portable potty/commode or other facility where Biofluid of the user is received and processed.

Similarly a method of monitoring the healthiness of the individual or individuals by the Smart Health Monitoring, Analysis and Remediation Tracking (SHMART) system based on Bio-fluid also is disclosed here. It is comprising of:
a) stepping in and approaching the facility by the user and pressing the service activation button;
b) starting the analysis of the terms of service once the button is pressed;
c) identifying the person who is subjected to evaluation of the health confidentially that is distinguishable by unique used id, if the user if id already exists;
d) creating a unique user id as placeholder, if not available;
e) Smart Id subsystem validates the identification of the user;
f) triggers the Monitoring and Diagnostics subsystem to facilitate collection of the Health Data paired up with the User Identification;
g) secures it in the Health Data Control subsystem that is used by Analytics subsystem; and
h) generating remediation actions that are tracked by the Remediation Tracking subsystem to ensure the individual user and the preventive healthcare system as a whole benefit from it.

### BRIEF DESCRIPTION OF DRAWING

These and other features, aspects and advantages of the present invention will become apparent to those skilled in the art upon reading the following detailed description of the preferred embodiments, in conjunction with the following accompanying drawings.

These and other features, aspects and advantages of the present invention will become apparent to those skilled in the art upon reading the following detailed description of the preferred embodiments, in conjunction with the following accompanying drawings.

FIG. 1 depicts the work flow of a Smart Health Monitoring, Analysis and Remediation Tracking (SHMART) system based on Bio-fluid showing the role of associated components according to the invention.

FIG. 2 enumerates the Physical Components of the SHMART system in the embodiment as a toilet facility

FIG. 3 shows SHMART system deployed as multi-unit according to an embodiment under the invention.

FIG. 3b shows SHMART system as an another embodiment under the invention as a Retrofitted Waterless Urinal FIG. 4 shows the method of Smart Identity subsystem of the SHMART system.

FIG. 5 depicts the method of the health data control subsystem of the SHMART system.

FIG. 6 shows the method of monitoring and diagnosing subsystem of the SHMART system.

FIG. 7 shows method the Analytics subsystem of the SHMART system.

FIG. 8 shows the method of Remedy Tracking subsystem of the SHMART system.

FIG. 9 shows an overview of the method the various Subsystem of the SHMART system interact with each other to ensure the knowledge and intelligence accumulated and utilized.

Wherein the SHMART system components which comprise of “Physical Unit Components” which are depicted as rectangles in solid line, namely 1. Biometric Identifiers; 2. IOT-Processor; 3. Sensors; 4. Macerator; 5. Piezo Electro-Mechanics; 6. Specimen Cartridges; 7. Diagnosis Equipment; 8. Supply Cartridges; 9. User Interface, and “Software Infrastructure Components” which are depicted as rectangles in dotted line, namely 10. Local DB; 11. Onboard Analytics; 12. IOT-Network; 13. BlockChain; 14. Bio-Info Wallet; 15. Cloud DB; 16. Centralized Analytics; 17. Federated & Distributed Analytics, to achieve the “Methods” subsystems depicted as ovals, namely 18. Smart Identification; 19. Health Data Control; 20. Monitoring & Diagnostics; 21. Analytics; and 22. Remedy Tracking for the User, Provider and Researchers of Preventive Healthcare depicted in dark rectangles.

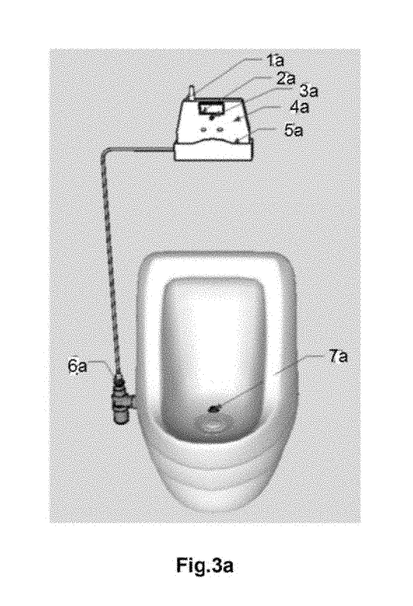

In FIG. 3a wherein 1a. IOT-Network whose antenna is shown; 2a. the User Interface (display and interaction buttons are shown); 3a. Biometric Identifier; 4a. IOT-Processor and Diagnostic Equipment located inside the housing; and 6a. The Piezo Electro-Mechanics and 7a. The Sensors all of which are attached to the urinal

### DETAILED DESCRIPTION OF THE INVENTION

The present invention provides state of the art smart bio-fluids diagnostic monitoring system and a method of monitoring healthiness. Said device is optimized for direct specimen capturing and mining with remote configuration and management & able to legally comply with personal health data control. Some of the characteristic designed features of the Smart Health Monitoring, Analysis and Remediation Tracking (SHMART) system according to our invention are as under:

SHMART is Designed to be “Individualized”:

Individual participation is a prime requirement to the success of any preventive healthcare system as increased participation helps compile more contextual data, which in turn helps the healthcare ecosystem thrive. SHMART is designed to encourage individual participation in several ways:
1. It requires minimal interaction of the individual to facilitate the diagnostic measurement. It is designed to be mostly inconspicuous to the user and work behind the scenes and user need not wear on person or puncture the body, charge the device or even synchronize data to ensure their information is secured.
2. It individually determines services to be rendered based on personal profile and system defined rules.
3. It provides instant gratification by its ability to provide results immediately.
4. In case of any evidence of match with a concern causing pattern, it is able to offer follow-up actions.
5. It organizes the compiled data in a personal-info wallet and allows the individual full control over non-aggregate personal and private information.

SHMART is Designed for “Mass” Implementation:

Mass implementation is a key requirement for effective Preventive Healthcare as it allows building up the critical volumes of Meaningful Facts and Rapid integration with Healthcare services and resources. SHMART integrates best into Mass deployment applications because of the following reasons:
1. It offers a lower cost (several orders of magnitude) to the alternative of deploying DLP in all regions especially in remote and rural areas.
2. It has built in and configurable facilities to become compliant and maintain compliance with the law of the jurisdiction.
3. It ensures Data exchanged and transmitted must meet the individual needs of privacy and public needs of security.
4. It assimilates several features such as a macerator that aids in water conservation and commercial value of aggregate data that in-turn that make it lucrative to deploy in Mass markets.

SHMART is Designed to Direct Collection of “BioFluids”:

The SHMART system monitors more health related attributes of the individual more accurately and at more consistent and regular intervals and more inconspicuously than any other available system or service. It is able to do so because it measures the data directly at the source. It is improves on

Diagnostic Lab Provider (DLP) and the Action Tracking Device (ATD) solutions in several ways:
1. It does not require a person or service to handle, store, and securely transport the BioFluid specimen to the DLP.
2. Due to physical and chronological proximity to the source of the BioFluid Specimen, it is able to capture several attributes that are not possible by DLP such as volumetric and olfactory measurements.
3. It allows multi-sample trending analysis without requiring in-patient service or multiple DLP visits.
4. The instantaneous nature of the SHMART system allows a graduated diagnosis on the same specimen i.e. preliminary patterns matches triggering deeper analysis.
5. Unlike the ATD solution, it measures the primary internal sources attributes rather the exterior sources. Chemical composition is a more complete and reliably early-indicator and professionally preferred compared to consumer level physical attributes.

SHMART is Designed to Make “Smart” Decisions:

This is the brains of the system that makes the SHMART system automated, extensible, deployable remotely and work behind the scenes. It is enabled by the combination of the microprocessor, communication and activator systems and that control the system in the following ways:
1. Manages the diagnostics component of system to determine and prioritize services to be rendered, verify the measurements conducted and assign higher sensitivity to certain resultant patterns.
2. Manages the configuration of system to provide services individualized to the user based on individuals personal profile and system defined rules.
3. Manages two-way communications with central databases and with regional providers and allows remote configuration.
4. Intelligently interfaces with user as and when needed.
5. Able to distinguish and identify among the multitude of users of the system.
6. Secures the personal information wallets and allows user to granularly grant access to the medical providers and other possible recipients.
7. Troubleshoots when system is dysfunctional or needs maintenance supplies.
8. Make system extensible so as to be able to add features and services in the future as per changing market demands and governing regulations.
9. Allows adding features to help improve appeal to mass deployment.

SHMART is Designed to Consistently and Contiguously do “Health Monitoring”:

Contiguous and consistent Health Monitoring is vital for preventive healthcare SHMART facilitates maintaining the contiguous of Health Monitoring by:
1. Automating the procedures and making functions seamless and inconspicuous.
2. Proactively managing maintenance and replenishment of supplies.
3. Facilitating maintain communication with various levels of players of the Healthcare ecosystem including DLPs, vendors, pharmacies and doctors.
4. Providing aggregate and authorized individual data for immediate remediation, medical research and drug efficacy tests.
5. Keeping the individual engaged by transparently providing benefits.

SHMART is Designed to Coordinate Preventive “Analysis”:

Analysis is the ultimate value-add provided by the SHMART system. Analysis is provided at various levels by the system direct and indirectly, and over time. It ensures maximum yield to benefit Preventive Healthcare by analyzing in 4 different ways:
1. Onboard Analysis is carried out immediately upon collection of the BioFluid data. It compares against available patterns and tolerance thresholds to diagnose preliminary or ingrained health concerns. It may also take user identity into account to initiate additional diagnosis, sample collections or offer follow-up actions based on the analysis.
2. Centralized Analysis This is the second and higher level of analysis that cumulates numerous Onboard Analysis received from a region and uses timeline analysis to decipher very-early patterns that indicated contagious decease or public health issues from water, air or other contaminants. It will also conduct more complex and elaborate pattern analysis (such as backward tracing for researching root-causes & helping identify possible antidote) with the help of higher processing power and access to larger database.
3. Federated Analysis is carried out in collaboration with SHMART partners associated to mutually benefit from the research.
4. Distributed Analysis is carried out by independent organizations that tap into the SHMART repository's aggregated and authorized individual data for social or commercial purposes of Healthcare.

In other words SHMART system is state of the art tool that can extend the human Life-span by spotting disease trends early, helping pharmaceuticals Customize research & eliminate duds sooner. It also reduces health care emergencies, and aids suffering populations making the world safer. It can also be deployed in a manner to become a financial platform to facilitate commercial support for social causes such as pharmaceutical research entities funding rural sanitary-management infrastructure.

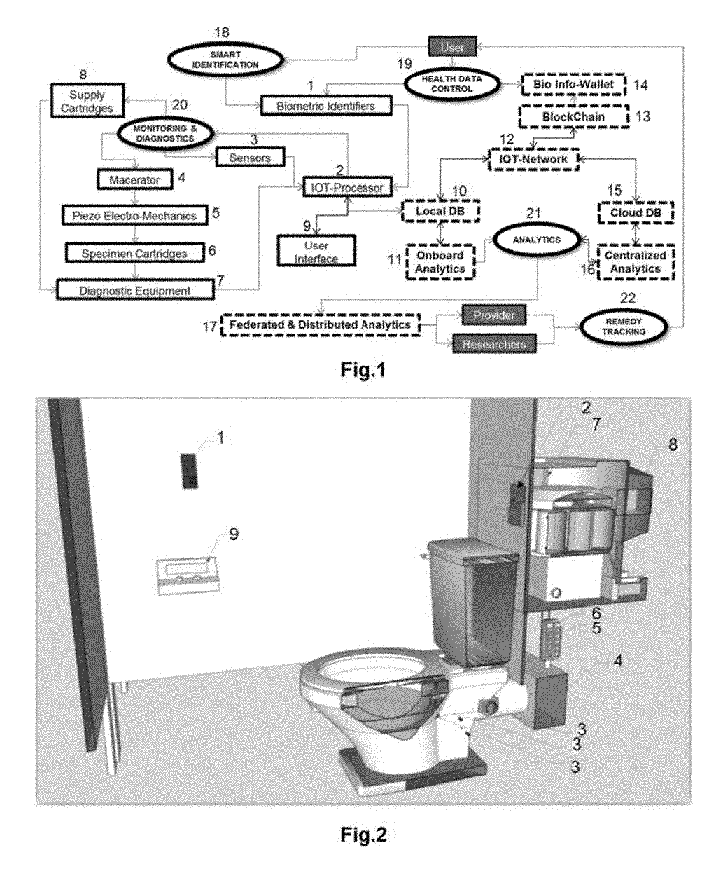

Over view of the work flow of a Smart Health Monitoring, Analysis and Remediation Tracking (SHMART) system based on Bio-fluid shows the role of associated components according to the invention is shown in FIG. 1. The functioning of the system according to the preferred embodiment is described here in brief.

The Biometric Identifiers of the SHMART system helps to identify the user of the SHMART system facility and record the information with the IOT-Processor which in-turn triggers the Monitoring and Diagnostics subsystem that activates Sensors and Diagnostic Equipment along with the dependent components namely the Supply Cartridges that ensure supply of raw materials required for diagnostics. The macerator provided there in extracts the BioFluid and enhances its consistency in readying it for processing in the diagnostic equipment and conveys it with the help of Piezo Electro-Mechanics to the Specimen Cartridges, wherefrom the BioFluid is sampled by the Diagnostic Equipment either in a single pass or multiple pass, based on the Diagnostic Plan for the particular user and returns the diagnostic information to the IOT-Processor. The IOT-Processor in turn records the diagnostic data in the Local Data Base, which then gets analyzed by Onboard Analytics to gather some preliminary health intelligence relating to the diagnosis, which after being interactively conveyed to the user along with the diagnostic information by the IOT-Processor gets uploaded for secure and highly-available storage to the Cloud DB through the IOT-Network, and simultaneously creates a record of the transaction in the BlockChain's Bio-Info Wallet belonging to the User. The Centralized Analytics uses it's more resourceful and sophisticated analytic power gained from access to the all the SHMART system units connected to the interconnected network and Federated & Distributed Analytics available through SHMART system associated Medical Providers and Researchers to narrow down a course of action to Remedy any healthcare concern relating to the diagnosis and Track the activity related to the Remedy in conjunction with the User and their repeated user of the SHMART system.

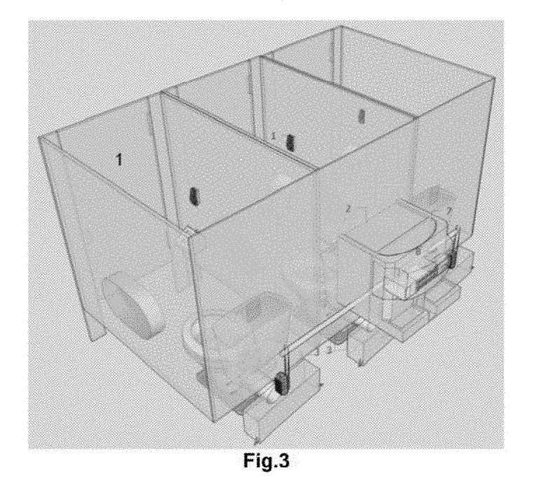

The Physical Components of the SHMART system according to an exemplary embodiment as a toilet facility is shown in FIG. 2. SHMART system deployed as multi-unit is shown in FIG. 3. Here a cluster of 3 toilet stalls are simultaneously deployed each with their own sensors, user interfaces macerators and other components yet share the diagnostic equipment and supply cartridges hence conserving costs and making feasibility more elaborate diagnostics tests.

The Smart Health Monitoring, Analysis and Remediation Tracking (SHMART) system based on Bio-fluid comprising of Biometric Identifiers (1) that automatically and seamlessly identify or validate the user of the SHMART system; IOT-Processor (2), a micro-processing computer that is designed for Internet-of-Things; a set of sensors selected (3) from Ambient sensors, Perfunctory sensors, Supply Sensors and Biometric; Macerator (4) to homogenize the consistency of the bio-fluids; Piezo Electro-Mechanic devices (5) to convey tightly-controlled quantities of fluids into the necessary SHMART components as necessary; Specimen Cartridges (6) for holding in-process BioFluids in preparation of diagnostics; Diagnosis Equipment (7) compatible for deploying in the SHMART system; Supply Cartridges (8): required by the diagnostic equipment; User Interface (9) that allows the user to interact with the System; Local database (10) an electronic store that holds the data; Onboard Analytics (11), a set of rules forming the 1st level of intelligence of the system; IOT-Network (12) that helps to communicate with rest of the network; BlockChain (13), a data security mechanism; Bio-Info Wallet (14), a software folder that contains a key to all the diagnostic information associated with a particular user; Cloud Data Base (15), Centralized Analytics (16), to the Onboard Analytics; Federated & Distributed Analytics (17); all of which are configured to work with semi portable potty/commode or other facility where biofluid of the user is received and processed.

FIG. 3a shows SHMART system as an another embodiment under the invention as a Retrofitted Waterless Urinal.

Here the SHMART system is retrofitted into an existing waterless urinal facility from a standard commercial manufacturer and several of the “Physical Unit Components” of SHMART system are integrated into single unit located above the urinal system including the 1a. IOT-Network whose antenna is shown; 2a. the User Interface whose display and interaction buttons are shown; 3a. Biometric Identifier which in this case is an iris scanner whose camera is co-located with the User Interface and the 4a. IOT-Processor and Diagnostic Equipment located inside the housing; and 6a. The Piezo Electro-Mechanics and 7a. the Sensors; are attached to the urinal

Whereas the other parts of the SHMART system namely the Macerator, the Specimen Cartridges and Supply Cartridges are not needed in this special instance of embodiment as the waterless system reduces the need to improve consistency; and Specimen and Supply Cartridge are not installed due to the simplified nature of diagnostics in this system

Whereas even more microscopic version of a SHMART system is conceptually envisaged in the future to be implemented into a tooth-cap or tooth-implant that integrates a microscopic processor with a long-life battery (to the order of 20 years or that may be recharged by the jaw action) and NFC communication that will be able convey fundamental saliva or mucus statistics to a smart device carried on the body will aid even better preventive health of weight control and oral health which recent studies have shown is related to major health issues such as heart deceases.

FIG. 4 shows the method of Smart Identity subsystem of the SHMART system, wherein upon user utilizing the SHMART system Analyzes Service Requirements level of user Identification and list of diagnosis that need to be conducted based on rules and priorities configured into the system and accordingly activates User Identification system which utilizes the various biometric and other means to identify an existing (previously known to the SHMART system) or new user to synchronize an existing Bio-Info Wallet or create a new one, and also creates the Diagnostic Plan & ID to trigger Monitoring & Diagnostic subsystem and upon receiving the diagnostic information and updating the Bio-Info Wallet and stores it in the Health Data Control subsystem where the Analytics subsystem uses the data to prime and update the User Identification information for future use and forwards any healthcare related action deemed necessary to be taken by the individual or general preventive healthcare to the Remedy Tracking subsystem to be managed.

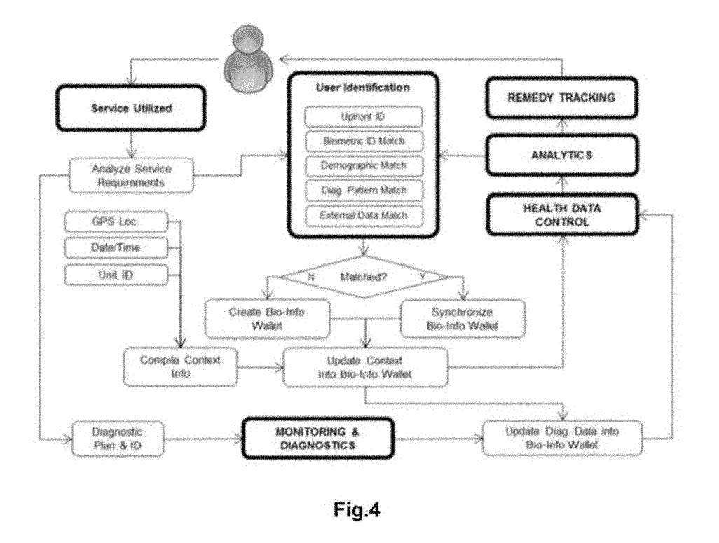

According to FIG. 5 the Diagnostic Data Update received from the Monitoring & Diagnostic module is stored in the Local DB and presented to the user as Personal Data by a Spot Report and if the user claims ownership of the data tags the data into the user's Bio-Info Wallet received from the BlockChain and uploads it to the highly secure and globally and instantly available Cloud DB as Cloud DB Private Data and if the user does not choose to take ownership of the data tags the data as Aggregate Data (without users Bio-Info Wallet information) and stores it as Cloud DB Aggregate Data

The Cloud DB secures the data beyond access to any unregistered (and hence invalid) 3^{rd} Parties and 3^{rd} Parties such as Medical Providers and Research Organizations, then who go through a verification process and acquire a 3^{rd} Party Identity Wallet and access to selective parts of the data by virtue of being authorized by the Contractual Authorization by the SHMART system or the Wallet Owner Authorization or Paid Authorization received from the user.

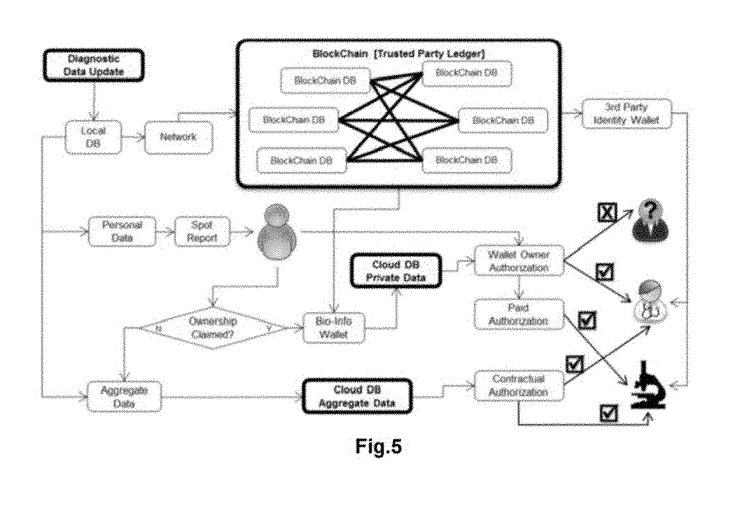

As shown in FIG. 6, the Monitoring & Diagnostics subsystem receives the Diagnostic Plan & ID from the Smart Identification subsystem and configures the Ambient Sensors, Perfunctory Sensors to collect ambient readings such as temperature, air and water quality and perfunctory readings such as Odor, PH Level, Mass, Volumetric and Optical of the BioFluid and also initiates the Supply Logistics to prepare the Diagnostic Equipment and based on the Supply Sensors information may alert the Equipment Maintenance which in-turn triggers maintenance through the Vendor Communications or Cleansing & Preparation to make the SHMART system Ready to Service, and the Diagnostic Equipment, which may vary widely depending on the deployment conducts various diagnostic tests for Infections, Nutritional Info, Gastroenterological or other Specialized/Custom Tests including possible DNA Host/Parasite Analysis and upon completion of the diagnostics updates the Bio-Info Wallet with such information which is then stored in the Health Data Control subsystem and progressed through Analytics and Remedy Tracking subsystem.

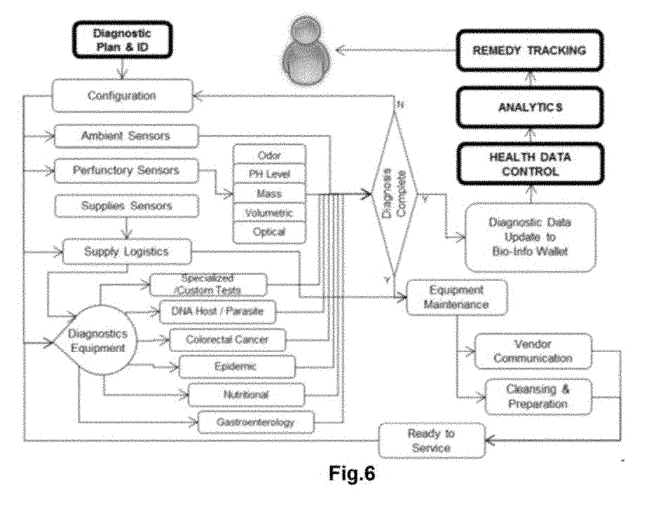

Method the Analytics subsystem of the SHMART system is shown in FIG. 7 wherein the IOT-Processor updates the Local DB and Cloud DB using the IOT-Network. The Onboard Analysis and Centralized Analysis use the Predefined Priorities, Known Patterns, Threshold of Facts, Identity of the User, Diagnostic Data and Sensor data to analyze for interesting facts related to preventive healthcare, which allows the Onboard Analysis to present the information to the user through a Spot Report and the user in-turn may act up the information and grant access to Authorized Personal Data to their Health Provider who in turn will access the more complete analysis coming out of the Centralized Analysis and the Aggregate Data within the Centralized Analysis being made available to other Medical Researchers, Social Organizations and Other Collaborators that conduct Distributed Analytics and Federated Analytics and increase the utility of the data in the Centralized Analysis for further improvement of Preventive Healthcare.

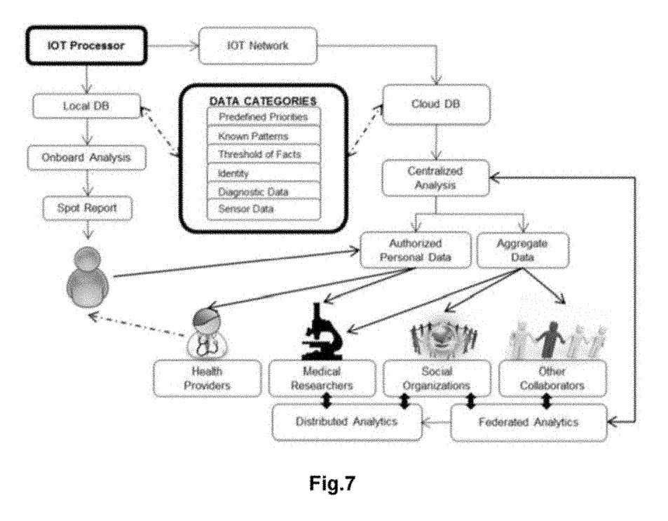

FIG. 8 shows the method of Remedy Tracking subsystem of the SHMART system. Here the analysis from the Onboard Analysis and Centralized Analysis are communicated to the user through SHMART User Interface, Consumer Device or SHMART Centralized Communication sothat the User can initiate Remediation using Healthcare Provider Communication that may be tracked by SHMART system and the Aggregate Data coming out of the Centralized Analysis can be accessed by Medical Researchers, Social Organizations and Other Collaborators of the SHMART system who in-turn will use the data for the preventive healthcare actions that can be tracked by the SHMART system.

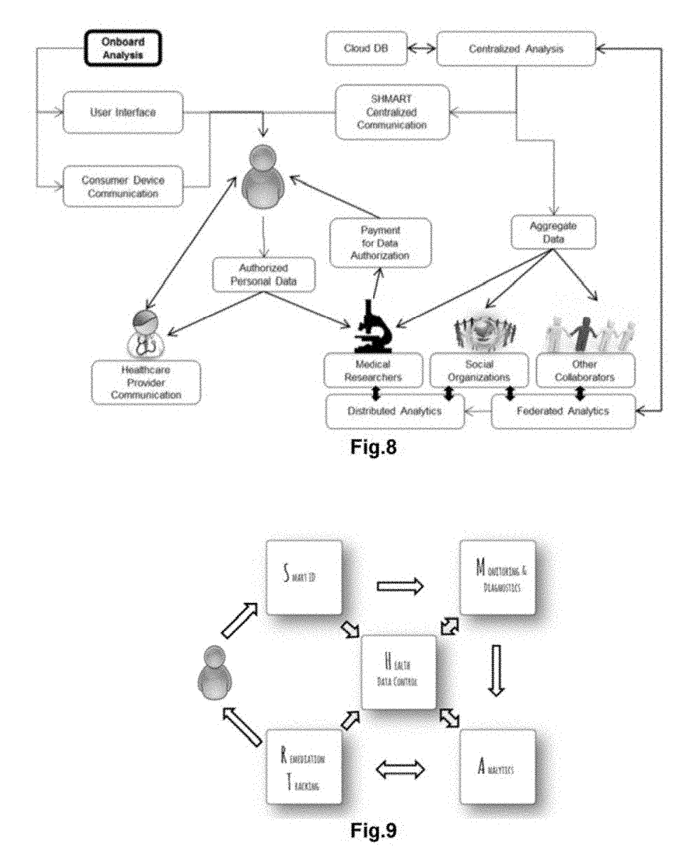

FIG. 9 shows an overview of the method by which various Subsystem of the SHMART system interact with each other to ensure the knowledge and intelligence accumulated and utilized, wherein the Smart Id subsystem validates the identification of the user and triggers the Monitoring and Diagnostics subsystem to facilitate collection of the Health Data paired up with the User Identification and secures it in the Health Data Control subsystem that is used by Analytics subsystem to generate remediation actions that are tracked by the Remediation Tracking subsystem to ensure the individual user and the preventive healthcare system as a whole benefit from it.

Components & Accessories of the SHMART system according to the invention are enumerated and defined here:
1. Biometric Identifiers: Biometric Identifiers are deployed to automatically and seamlessly identify or validate the user of the SHMART system without requiring the user to provide manually enter it or use any hard evidence such as swipe card. The system may be deployed in a myriad of combinations including finger printing during opening of facility, activating flush level, or with a physical console installed on side, iris scan system or other system
2. IOT-Processor: IOT-Processor (also known as Computer) is a micro-processing computer that is designed for Internet-of-Things in that it can sustain on low-power and able to communicate with IOT Networks. So it is able to work for prolong periods on battery and appropriate for remote locations. It provides considerable computing power and sufficient network bandwidth, however depending on deployment type, level of diagnostics & local analytics provided and network infrastructure, compact-fan-less small form-factor or standard computers may be used.
3. Sensors: Sensors deployed in the SHMART system range widely including Ambient, Perfunctory and Supply Sensors in addition to the Biometric sensors discussed earlier. Ambient Sensors record contextual information including quality and quality of intake water, local temperatures and air-quality etc. that may be relevant to the Analysis. Perfunctory Sensors record initially obvious characteristics of the biofluids including mass, volume, odor, Acidity or PH levels and optical. Supply sensors are those that gauge the level of consumable supplies required by the diagnostic equipment in order to proactively replenish them so as to maintain the utility of the system.
4. Macerator: Macerator has multiple functions. It primarily helps homogenize the consistency of the bio-fluids; but it also aids in building up the downstream pressure flowing through the system to aid water conservation and cleansing the system. Additionally it's characteristic of lowering the upstream pressure along with a toilet seat designed may be used to deliver a suppository effect to the user.
5. Piezo Electro-Mechanics: Piezo Electro-Mechanics devices are used to convey tightly-controlled quantities of fluids into the necessary SHMART components as necessary. It is directly controlled by the IOT Processor to transport bio-fluids, diagnostic supplies, cleaning and drying agents between SHMART components—Macerator to Specimen Cartridges to Diagnostic Equipment and Cleaning apparatus.
6. Specimen Cartridges: Specimen Cartridges are specially designed for holding in-process BioFluids in preparation of diagnostics and may vary by the deployment type of the SHMART system. They hold the specimen in a matrix of predefined consistency so as to allow selected, multiple and repeat diagnostic tests and thus allow flexibility to configure & revise the diagnostic plan on an as needed basis.
7. Diagnosis Equipment: Diagnosis Equipment deployed in the SHMART system varies through a wide range depending on the deployment purpose, level and sophistication of targeted services. However the standard requirement is that the diagnostic equipment must allow automated deployment and includes these features—Electronic reporting that can be directly read by the IOT Processor, ability to be electronically configured and setup, able to automate the feed of supplies and allow fitment of sensors that electronically update status of supplies etc.
8. Supply Cartridges: These hold the supplies required by the diagnostic equipment and are designed to and automatically feed the supplies based on instructions from the computer.
9. User Interface: The User Interface allows the user to interact with the System in a number of ways including selecting services required; obtaining a spot report; updating user identity info; viewing disclaimers or other legal information; authorizing data usage etc. It may also be used for system maintenance. The mode of communication also may vary widely from simple LED indicators, to visual electronic display, electronic communication with consumer devices with protocols such as Bluetooth and NFC. Depending on the deployment type it may also include printing facility to provide a hardcopy of the interaction.

Technology infrastructure associated with Physical Unit are as follows:
10. Local DB: The local database is an electronic store that holds the data in preparation to be uploaded to the Cloud DB and it compiles all readings from Sensors, Diagnostic Equipment, Usage Data, User Data, System status, input data and reports of the Onboard Analytics. Depending on the deployment type it may also contain downloaded information from the Centralized Analytics necessary for User Interface as part of the Remediation Tracking. The local DB may be software hosted on the same board as the IOT-Processor or may be implemented an separate but attached hardware. In either case its data is secured and encrypted.
11. Onboard Analytics: This is a set of rules forming the 1st level of intelligence of the system. It considers all the predefined priorities, known patterns and threshold of facts and the data in the local DB to infer other foretelling facts and any corresponding action items that may be useful to the users, their medical providers. The Onboard Analytics is usually implemented as software with the IOT-Processor but like the Local DB may be on attached hardware.
12. IOT-Network: This helps the individual SHMART system communicate with rest of the network especially the Cloud DB. It may also help the User Interface communicate with consumer devices with protocols such as Bluetooth, NFC and IP based Networks. This IOT system is capable of communicating over long ranges with low power antenna (which may not necessarily be visible externally) so as to conserve energy and allow long-term unattended operation of the SHMART system. Depending on the deployment model, this may also be implemented on WIFI or IP network.
13. Block Chain: This is a data security mechanism implemented as software-infrastructure that serves to compile and keep track of set of private health data facts collected by the SHMART system at each interaction of each user. It allows users to take ownership of such data and selectively authorize its distribution based upon the User's personal requirements.
14. Bio-Info Wallet: This is a software folder that contains a key to all the diagnostic information associated with a particular user. Whereas the actual diagnostic data is stored in the Cloud DB the references to the several datasets of that particular user is contained in a bio-info wallet. In the SHMART system information is only transferred between Bio-Info Wallets. So Medical providers, research organizations and any other entity requiring access to the User's Bio-Info Wallet needs to possess their own Bio-Info Wallet prior to receiving the user's data. Thus the sensitive information is secured as the identity of the receiver/consumer of the data will be traceable by the User.
15. Cloud DB: This is an Information Technology infrastructure that allows massive amounts of data to be hosted with centralized control yet being rapidly accessible from any part of the network. It provides a cost-effective, secure, scalable and flexible infrastructure to the users and SHMART system administration and consumer organizations.
16. Centralized Analytics: This is similar in composition to the Onboard Analytics in that it accesses all the sensors, diagnostics and user information along with a set of rules to develop alerts to understand trends that will be useful for Healthcare. However the Centralized Analytics does it on a global scale and accounts for every SHMART system connected. The Analytics it allows are drastically complex view.
17. Federated & Distributed Analytics: Federated Analytics is similar to Centralized Analytics but differs on the fact that is conducted by external partners that collaborate within the SHMART ecosystem and so may have access to richer knowledgebase to infer the analysis. Such extended analysis is typically circled back to the Centralized Analytics, thus mutually benefiting the organizations and the end user. Distributed Analysis on the other hand is conducted by 3rd Parties who commercially acquire the Bio-Info from the SHMART system without an obligation to circle back the conclusions of their Analytics.

According to the invention, a method of monitoring the healthiness of the individual or individuals by the Smart Health Monitoring, Analysis and Remediation Tracking (SHMART) system based on Bio-fluid comprising:
a) stepping in and approaching the facility by the user and pressing the service activation button;
b) starting the analysis of the terms of service once the button is pressed;
c) identifying the person who is subjected to evaluation of the health confidentially that is distinguishable by unique used id, if the user if id already exists;
d) creating a unique user id as placeholder user id, if not available;
e) smart Id subsystem validates the identification of the user;
f) triggers the Monitoring and Diagnostics subsystem to facilitate collection of the Health Data paired up with the User Identification;
g) secures it in the Health Data Control subsystem that is used by Analytics subsystem; and
h) generating remediation actions that are tracked by the Remediation Tracking subsystem to ensure the individual user and the preventive healthcare system as a whole benefit from it.

Smart Identification which is the method of ensuring that the identity of the user whose BioFluids are sampled and diagnosed is tracked in a way to provide transparency and traceability required for healthcare and associated research and provide opportunity for commercialization of the data & intelligence resultant from the process, yet allowing the user to maintain ownership and privacy of their personal confidential data. The identification of the user of the facility is captured upfront by cross-verification from several foolproof means (e.g. Biometric tools and physical sign-in) and paired up with the associated BioFluid analysis reports by a unique identifier known as a Bio-Info Wallet that is generated and recorded by a BlockChain mechanism that cannot be manipulated and protected from error due to being authenticated by boundless amount of distributed BlockChain services and that the user is afforded the option of pairing the BioFluid diagnostics with their Bio-Info Wallet retroactively after repeated use of the facility by the user all of which can be achieved by a set of components and methods designed into the SHMART system including:
1. Analyze Service Requirements: SHMART system is designed to be selectively conduct diagnostics depending on the specific requirement of the users rather than incur the resource costs of conducting all tests for all users. Analyze Service Requirement is the method of determining which diagnostics to conducted on the current user by analyzing the set of system rules configured into the system by the deploying agency, the situational context (e.g. U.S. Federal Emergency Management Agency or (FEMA) may deploy a set of SHMART system in a Flood emergency to look for Diarrhea symptoms rather than influenza) and any known data on the individuals susceptibility to that ailment being sensitively screened. The sensitivity of screening for other ailments may be turned lower in such a situation.
2. Diagnostic Plan & ID: This is the final output “Analyze Service Requirements” function which is encoded with a unique key (the Diagnostic Plan ID) which is sent to the diagnostic equipment configuration and tracked by the Data Control so as to provide information to the Analytics and Action Coordination methods for Analysis and further action.
3. Monitoring & Diagnostics: See information on Monitoring & Diagnostics section.
4. Upfront ID: This form of User Identification is applicable when users of the SHMART system chooses to manually identify themselves using a physical method such as entering a User ID into the User Interface.
5. Biometric ID Match: This is a form of User Identification where Biometric Identification tools such as finger-print analysis, iris scan or other means are used to either primarily identify the user or to cross verify the identification of the user of the SHMART system.
6. Demographic Match: This form of User Identification is used when the users chooses not to identify themselves, however a partial demographic identity of the user is possibly to be identified for the purposes of aggregated data used for research without which the relevance of the data captured is practically rendered useless. Such demographic identity may be established with low cost DNA tests conducted on the BioFluid sample. Such identification may be conducted at a later phase rather than at the time of service.
7. Diag. Pattern Match: This is another type of user identification that is possible to corroborate the identity of the user based on the diagnostic patterns currently observed with those conducted by another service that is part of the SHMART system.
8. External Data Match: This method of user identification is possible by corroborating the partial identity captured by the SHMART system by externally available user and usage data e.g. from social network other such data.
9. Decision if Match Found: This is a contingent function that depends on the fact if the Identity of User is found within the SHMART system by any of the means described above. If the user is not identified in spite of all the options available above and new Bio-Wallet is created with the any of the identification available or just as a as a placeholder in anticipation of such identifying information being available subsequently.
10. Creating a Bio-Info Wallet: This is a method of creating a unique identification number within the BlockChain infrastructure that as described elsewhere in this document, is a secure, fool-proof, non-repudiating system designed for allowing privacy and control to it owner.
11. Synchronizing a Bio-Info Wallet: This is the process of updating the Bio-Info Wallet of a user who currently has an existing Wallet with any new informational update captured by the User Identification.
12. GPS Location: This is a sensor installed into the SHMART system that allows it to determine the exact location at the time the user used the system. This is necessary to analyze if any interesting diagnostic intelligence found in the BioFluid may be geographically related such a local water contamination.
13. Date/Time Stamp: This is a sensor that provides chronological relevance and evidence to any interesting diagnostic intelligence found in the BioFluid of the user.
14. Unit ID: This Identification of the specific unit of the SHMART system network is necessary to enhance the accuracy of the GPS location Info as several units may coexist in the same proximity and that may not be distinguished by the GPS system. It is also used to corroborate any calibration issues with the unit itself.
15. Compile Context Info: This is function to compile the GPS Location, Unit ID and Chronological sequence into a unique identifier that securely codifies that information as well as reduces the amount of data necessary to be tracked throughout the system.
16. Updating Context into Bio-Info Wallet: This is the function of adding the Context Info in the Bio-Info Wallet to aid validating the specific instance of diagnostic data monitored for the user's data analysis.
17. Update Diag. Data into Bio-Info Wallet: This is the function of adding Diagnostic Data into the Bio-Info Wallet of the user.
18. Health Data Control: See information on Health Data Control section.
19. Analytics: See information on Analytics section.
20. Remedy Tracking: See information on Remedy Tracking section.

Health Data Control is the method of ensuring the user of the facility contributing the Bio-Fluid is allowed maximum possibility to claim the ownership of the Health Data and the SHMART system can collaborate with the owner of the data to maximize its utility for preventive healthcare purposes and to allow secure storage and transmission of the Health Data as it flows through various actors as necessary for the health care ecosystem to work towards an efficient preventive healthcare system. The use of a Bio-Info Wallet consolidates all the references to data relating to a particular owner of the data and any data tractions are managed through BlockChain mechanism infrastructure that cannot be manipulated and is protected from errors due to being authenticated by boundless amount of distributed BlockChain services and the SHMART system has control over Aggregated Data generated from contiguous monitoring and to manage all data in a global-scale, reliable and high-availability environment and a with a technology resistant to being obsolete, all of which can be achieved by a set of components and methods designed into the SHMART system including:
1. Local DB: This is a database located on each unit of the SHMART system network or may also be deployed as a single instance for an entire cluster of units located in close proximity. This database stores in a secure and encoded manner all the relating to the user, sensors, diagnostics, all software necessary for running, configuration and maintenance of the system and configuration and most importantly the business rules necessary for Onboard Analysis of the diagnostic data and user interaction. However for performance, additional security reasons and to reduce the overall cost of the system, the range and amount of data left in the Local DB is balanced based on determined by the network configuration and bandwidth available to the system and the frequency and usage patterns of the users accessing the unit or cluster of unit.
2. Personal Data: Personal Data is the most recent sensor and diagnostic data captured by the SHMART system and it is only available to be viewed through the User Interface until the system is able to confidently infer that the data will be viewed by the same user that contributed the BioFluid sample. In cases that confidence gets distorted by the change in status of the system (such as the user exiting the unit) the information is removed from the User Interface for security purposes and is stored in the local DB in an encrypted form and categorized as “Aggregate Data”. For the Personal Data to be system become available through the Users Bio-Info Wallet, User will need to claim it through the User Interface by claiming ownership (selecting a button).
3. Spot Report: This is a direct display (or print out) of the users diagnostic report of the Personal Data and is available only prior to the user exists the system.
4. Network: This helps the individual SHMART system communicate with rest of the network especially the Cloud DB. It may also help the User Interface communicate with consumer devices with protocols such as Bluetooth, NFC and IP based Networks. This IOT system is capable of communicating over long ranges with low power antenna (which may not necessarily be visible externally) so as to conserve energy and allow long-term unattended operation of the SHMART system. Depending on the deployment model, this may also be implemented on WIFI or IP network.
5. BlockChain: This is a data security mechanism implemented as software-infrastructure that serves to keep track of set of private health data facts collected by the SHMART system at each interaction of each user. It allows users to take ownership of the such data and selectively authorize it's distribution based upon the User's personal requirements.
6. Bio-Info Wallet: This is a software folder that contains a key to all the diagnostic information associated with a particular user. Whereas the actual diagnostic data is stored in the Cloud DB the references to the several datasets of that particular user is contained in a bio-info wallet. In the SHMART system information is only transferred between Bio-Info Wallets. So Medical providers, research organizations and any other entity requiring access to the User's Bio-Info Wallet needs to possess their own Bio-Info Wallet prior to receiving the user's data. Thus the sensitive information is secured as the identity of the receiver/consumer of the data will be traceable by the User.
7. Wallet Ownership Claim: Upon claiming the personal data by the user a Bio-Info Wallet is created by the system, provided the user does not already have one, in which case the existing Wallet is used.
8. Cloud DB Private Data: The personal data after being claimed by the current users and tagged as attached with a specific Bio-Info Wallet is then stored in a more secure location of the Cloud DB. Once the personal data is classified as “Private Data” it will be in the ownership of the user. The user is then able to allow access to it at a very granular level of control to any party having a valid “3rd Party Identity Wallet”.
9. Aggregate Data: Any data handled by the SHMART system that is not classified as Private data is categorized as Aggregate Data and is in the control and ownership of the SHMART administrator and may be used for preventive healthcare or similar social benefit.
10. Cloud DB Aggregate Data: It is part of the diagnostic, sensor and other Aggregate Data located within the Cloud DB that cannot be considered Private Data.
11.3rd Party Identity Wallet: This is similar to the Bio-Information Wallet but is associated with the consumer-organizations of the data such as Medical Providers, Medical Researches, Government and Social Organizations. The purpose of it is to identify the receiver of the Private Data authorized by the user or Aggregate Data authorized by the SHMART administrator and maintain accountability.
12. Wallet Owner Authorization: This is method for the users to selectively allow access to the 3rd Parties for serving the medical purposes of the user.
13. Paid Authorization: This is method for the users to selectively allow access to the 3rd Parties as donation for direct (monetary) or indirect (social causes or other) benefit to user.
14. Contractual Authorization: This is the method that SHMART system allows access to 3rd Parties to further the cause of preventive healthcare.

Monitoring & Diagnostics is a method of maximizing the automation necessary for the capture of meaningful facts from the BioFluid sample obtained by the SHMART system from the facility user and handling biofluids from capture, preparation for and conduction of diagnostics, archiving and ultimately to proper disposal in a safest manner possible in compliance to healthcare standards and regulations and allowing autonomous maintenance of the system so as to minimize costs and downtime of the SHMART system and fastest turnaround time between services. Use of Sensors are deployed to capture all relevant and possible data from ambience factors of the local environment to levels of supply materials required for diagnostics and to record Perfunctory information of BioFluids and a series of diagnostic equipment is setup to maximize sensitivity to capture any traces of infection or other diagnostically important factors that aid remediation and preventive healthcare and document the results from the Sensors and Diagnostic securely into the Data Control mechanism, all of which can be achieved by a set of components and methods designed into the SHMART system including:
1. Configuration: This is the set of instructions sent to the Diagnostic Equipment, Supply Logistics and Sensors in order to prepare them to conduct the specific set of diagnostics defined by the Diagnostic Plan.
2. Ambient Sensors: Ambient Sensors record contextual information including quality and quality of intake water, local temperatures and air-quality etc. that may be relevant to the Analysis.
3. Perfunctory Sensors: Perfunctory Sensors record initially obvious characteristics of the biofluids including mass, volume, odor, Acidity or PH levels and optical.
4. Supplies Sensors: Supply sensors are those that gauge the level of consumable supplies required by the diagnostic equipment in order to proactively replenish them so as to maintain the utility of the system.
5. Diagnostics Equipment: Diagnosis Equipment deployed in the SHMART system vary a wide range depending on the deployment purpose, level and sophistication of targeted services. A limited set of diagnostic tests are conceptually depicted in the above referenced figure, however in reality the range of tests will vary significantly enough based on the deployment model. The standard requirement is that the diagnostic equipment must allow automated deployment and includes these features Electronic reporting that can be directly read by the IOT Processor, ability to be electronically configured and setup, able to automate the feed of supplies and allow fitment of sensors that electronically update status of supplies etc.
6. Diagnosis Complete?: This is a contingent step designed into the system to allow multiple runs of the diagnostic equipment on the same BioFluid. This may be necessary to ratify an observed fact, or to modify the sensitivity to analyze certain probable dependent/secondary investigative facts and based on initial findings or to economize by limiting the diagnosis on the secondary investigative factors to only such cases that meet a relevant pattern. Note that the Diagnosis Complete also communicates status (including error messages) to Equipment Maintenance to be addressed in case of need.
7. Update Diagnostic Data into Bio-Info Wallet: This is the function of adding Diagnostic Data into the Bio-Info Wallet of the user.
8. Supply Logistics: This step is designed to ensure the diagnostic equipment required to be activated by the specific “Diagnostic Plan & ID” is primed with the necessary raw materials & supplies in order to execute the Diagnostic Plan successfully. In addition based on the readings of the “Supplies Sensors” it may send a request to the Equipment Maintenance function to replenish any supplies that may be running low.
9. Equipment Maintenance: Based on the Supply Logistics and status of “Diagnosis Complete?” the Equipment Maintenance communicates to vendors or maintenance personal if attention is required for additional supplies or address maintenance or calibration issues.
10. Vendor Communication: Vendor communication initiated by the “Equipment Maintenance” may include requests for replenishment of supplies, maintenance requests or calibration.
11. Cleansing & Preparation: Diagnostic Equipment has specific needs for cleansing after diagnostic runs, and steps to prepare the equipment for subsequent runs. This step attempts to automate such requests wherever possible or would generate a service request to be conducted manually by collaborating with the Vendor Communication.
12. Ready to Service: This step is to ensure the end state of the Equipment Maintenance, Vendor Communication and Cleansing & Preparation have resulted in readying the SHMART system for the next required service. In case of any exception it will raise an emergency maintenance request so as to minimize any downtime of the system. It may also selectively open certain diagnostics for next required service while denying other tests depending on the state of readiness of the system.

Analytics is the method of reaping maximum benefits of preventive healthcare and societal good from the sum of the monitoring & diagnostics data available to the SHMART system and to benefit the user of the facility while collaborating with all possible entities vested in achieving an efficient preventive healthcare and leveraging of knowledge within such entities to the fullest extent possible and positioning the acquired intelligence in a way to harvest it for current and future benefits. Here the use of Onboard Analysis allows an incentive and immediate gratification to the user of the system to encourage repeated participation in the SHMART system and use of Centralized Analysis to create one formidable repository [Cloud DB] of knowledge for benefit of preventive healthcare and using Federated Analytics and Distributed Analytics to enhance collaboration of development of such preventive healthcare knowledge and contribution to the Centralized Analysis and Cloud DB, all of which can be achieved by a set of components and methods designed into the SHMART system including:
1. Local DB: The local database is an electronic store that holds the data in preparation to be uploaded to the Cloud DB and it compiles all readings from Sensors, Diagnostic Equipment, Usage Data, User Data, System status, input data and reports of the Onboard Analytics. Depending on the deployment type it may also contain downloaded information from the Centralized Analytics necessary for User Interface as part of the Remediation Tracking. The local DB may be software hosted on the same board as the IOT-Processor or may be implemented an separate but attached hardware. In either case it's data is secured and encrypted.
2. Onboard Analysis: This is a set of rules forming the 1st level of intelligence of the system. It considers all the predefined priorities, known patterns and threshold of facts and the data in the local DB to infer other foretelling facts and any corresponding action items that may be useful to the users, their medical providers. The Onboard Analytics is usually implemented as software with the IOT-Processor but like the Local DB may be on attached hardware.
3. IOT Network: This helps the individual SHMART system communicate with rest of the network especially the Cloud DB. It may also help the User Interface communicate with consumer devices with protocols such as Bluetooth, NFC and IP based Networks. This IOT system is capable of communicating over long ranges with low power antenna (which may not necessarily be visible externally) so as to conserve energy and allow long-term unattended operation of the SHMART system. Depending on the deployment model, this may also be implemented on WIFI or IP network.
4. Cloud DB: This is an Information Technology infrastructure that allows massive amounts of data to be hosted with centralized control yet being rapidly accessible from any part of the network. It provides a cost-effective, secure, scalable and flexible infrastructure to the users and SHMART system administration and consumer organizations.
5. Centralized Analysis: This is similar in composition to the Onboard Analytics in that it accesses all the sensors, diagnostics and user information along with a set of rules to develop alerts to understand trends that will be useful for Healthcare. However the Centralized Analytics does it on a global scale and accounts for every SHMART system connected. The Analytics it allows are drastically complex view.
6. Authorized Personal Data: This is the personal data attached to a particular Bio-Info Wallet authorized by the owner of Bio-Info Wallet to be accessed by a 3rd Party Wallet for the medical remediation, direct or indirect purposes of themselves.
7. Aggregate Data: This is the non-personal data that is summarized and aggregated so as to remove any references from the diagnostic data to an individual person to whom the Bio-info may related to.
8. Health Providers: These are medical providers that provide the medical remediation services directly to the users.
9. Medical Researchers: These are commercial organizations involved in use the data from SHMART system for general medical research and not relating directly to the users.
10. Social Organizations: These are social organizations involved in use the data from SHMART system for general medical research that may or may not relating directly to the users.
11. Other Collaborators: Are partners of the SHMART system that have a common interest and a formal mutual relationship with the SHMART systems goals of developing an efficient preventive healthcare system.
12. Federated & Distributed Analytics: Federated Analytics is similar to Centralized Analytics but differs on the fact that is conducted by external partners that collaborate within the SHMART ecosystem and so may have access to richer knowledgebase to infer the analysis. Such extended analysis is typically circled back to the Centralized Analytics, thus mutually benefiting the organizations and the end user. Distributed Analysis on the other hand is conducted by 3rd Parties who commercially acquire the Bio-Info from the SHMART system without an obligation to circle back the conclusions of their Analytics.

Remedy Tracking is a method to ensure the individual user using the SHMART facility as well as the society as a whole benefit from the knowledge and intelligence accumulated by the SHMART system and demonstrate such benefit in the form medical attention received by the individual or progress in the medical field towards preventive healthcare and that is especially evident in averting healthcare calamities in nascent stage or improving triage operations when necessary. The use of collaborative communication within the SHMART system facilitates tracking the interaction among the individual users, their corresponding medical providers, commercial and social medical research and service organizations and documenting such communication within the Cloud DB, all of which can be achieved by a set of components and methods designed into the SHMART system including:
1. User Interaction: The User Interface allows the user to interact with the System in a number of ways including selecting services required; obtaining a spot report; updating user identity info; viewing disclaimers or other legal information; authorizing data usage etc. It may also be used for system maintenance. The mode of communication also may vary widely from simple LED indicators, to visual electronic display, electronic communication with consumer devices with protocols such as Bluetooth and NFC. Depending on the deployment type it may also include printing facility to provide a hardcopy of the interaction.
2. Consumer Device Communication: Consumer Devices can be either a smart phone that facilitates users to communicate with the SHMART system through an App or a web browser that communicates via a website to dispense all such information available to the SHMART User Interface or a more sophisticated interaction of the Cloud DB.
3. Authorized Personal Data: This is the personal data attached to a particular Bio-Info Wallet authorized by the owner of Bio-Info Wallet to be accessed by a 3rd Party Wallet for the medical remediation, direct or indirect purposes of themselves.
4. Healthcare Provider Communication: This is communication and interaction conducted through the SHMART system by the user with medical providers that provide the medical remediation services directly to the users.
5. SHMART Centralized Communication: This is communication and interaction conducted between the central database of the SHMART system and the user that may or not include 3rd Parties as part of the communication.
6. Aggregate Data: This is the non-personal data that is summarized and aggregated so as to remove any references from the diagnostic data to an individual person to whom the Bio-info may related to.
7. Medical Research Organizations: These are commercial organizations involved in use the data from SHMART system for general medical research and not relating directly to the users.
8. Social Organizations: These are social organizations involved in use the data from SHMART system for general medical research that may or may not relating directly to the users.
9. Other Collaborators: Are partners of the SHMART system that have a common interest and a formal mutual relationship with the SHMART systems goals of developing an efficient preventive healthcare system.

The main uses of the instant invention are as under:

| USAGE | OPTIONS TO BE DEPLOYED |
|---|---|
| Senior Housing | Customized of test suits |
| | Handicap access & support system |
| Poor | Self-Contained with Solar-Power, |
| Neighborhood | Water Cistern, Septic Tank |
| Infrastructure | High-Volume, Multi-unit Housing |
| Development | Maintenance Route Truck access |
| | Batch mode data transmission |
| Self-Serve Kiosks | Spot Communication using Bluetooth, |
| | Near Field & Hardcopy and or |
| | Electronic screen. |
| | Electronic payment management |
| | Self-Cleansing attachments |
| Hospital/Medical | Spot Customizable Test |
| Provider Facilities | configurations |
| | FDA Compliance Package |
| Drug Research | Lower-cost per Mass Research Unit |
| Facilities | Customizable User Interaction for |
| | Detailing, Disclaimers and additional |
| | user input/feedback. |
| | FDA Compliance Package & |
| | Anonymously Traceability |

In other words the smart bio-fluids diagnostic monitoring system is a tool that can extend the human Life-span by spotting disease trends early, helping pharmaceuticals Customize research and eliminate duds sooner. It also reduces health care emergencies, and aids suffering populations making the world safer.

It can also become a financial tool to facilitate commercial support for social causes such as pharmaceutical research entities funding rural waste-management infrastructure.

Further said smart bio-fluids diagnostic monitoring system are not cost prohibitive unlike traditional diagnostic labs available through medical providers when used for regular monitoring. Even it can be seen that the smart health monitoring devices hitherto used for health monitoring applications are limited to superficial monitors. Similarly dietary monitoring systems used at present are also relying on manual and subjective user input.

We have brought out the novel features of the invention by explaining some of the preferred embodiments under the invention, enabling any person skilled in the art to understand and visualize our invention. It is also to be understood that the invention is not limited in its application to the details set forth in the above description or illustrated in the drawings. Although the invention has been described in considerable detail with particular reference to certain preferred embodiments thereof, variations and modifications can be effected within the spirit and scope of the invention as described herein above and as defined in the appended claims.

I claim:

1. A Smart Health Monitoring, Analysis and Remediation Tracking (SHMART) system based on Bio-fluid comprising of Biometric Identifiers (1) that automatically and seamlessly identify or validate the user of the SHMART system; IOT-Processor (2), a micro-processing computer that is designed for Internet-of-Things; a set of sensors selected (3) selected from Ambient sensors, Perfunctory sensors, Supply Sensors and Biometric sensors; Macerator (4) to homogenize the consistency of the bio-fluids; Piezo Electro-Mechanic devices (5) to convey tightly-controlled quantities of fluids into the necessary SHMART components as necessary; Specimen Cartridges (6) for holding in-process BioFluids in preparation of diagnostics; Diagnosis Equipment (7) compatible for deploying in the SHMART system; Supply Cartridges (8) required by the diagnostic equipment; User Interface (9) that allows the user to interact with the System; Local database (10) an electronic store that holds the data; Onboard Analytics (11), a set of rules forming the 1st level of intelligence of the system; IOT-Network (12) that helps to communicate with rest of the network; BlockChain (13), a data security mechanism; Bio-Info Wallet (14), a software folder that contains a key to all the diagnostic information associated with a particular user; Cloud Data Base (15), Centralized Analytics (16); and Federated & Distributed Analytics (17); all of which are configured to work with semi portable potty/commode or other facility where Biofluid of the user is received and processed.

2. The system as claimed in claim 1, wherein it is retrofitted into an existing waterless urinal facility having a) IOT-Network; b) User Interface with display and interaction buttons; Biometric Identifier preferably an iris scanner whose camera is co-located with the User Interface and IOT-Processor and Diagnostic Equipment located inside the housing while the Piezo Electro-Mechanics and the Sensors are attached to the urinal.

3. The system as claimed in claim 1, wherein Biometric Identifiers are the one deployable automatically and seamlessly to identify or validate the user without requiring the user to manually enter or use any hard evidence such as swipe card and as such the system is deployable in a myriad of combinations including finger printing during opening of facility, activating flush level, or with a physical console installed on side, iris scan system or other system.

4. The system as claimed in claim 1, wherein IOT-Processor also known as Computer, is a micro-processing computer that is designed for Internet-of-Things in that it sustains on low-power and able to communicate with IOT Networks and as such able to work for prolong periods on battery and is appropriate for remote locations.

5. The system as claimed in claim 1, wherein a) Ambient Sensors record contextual information including quality and quality of intake water, local temperatures and air-quality that is relevant to the Analysis; b) Perfunctory Sensors record initially obvious characteristics of the biofluids including mass, volume, odor, Acidity or PH levels and optical; c) Supply sensors are those that gauge the level of consumable supplies required by the diagnostic equipment in order to proactively replenish them so as to maintain the utility of the system and d) the Biometric sensors.

6. The system as claimed in claim 1, wherein Macerator primarily helps to homogenize the consistency of the bio-fluids and also aids in building up the downstream pressure flowing through the system to aid water conservation and cleansing the system.

7. The system as claimed in claim 1, wherein Piezo Electro-Mechanics, devices used to convey tightly-controlled quantities of fluids into the necessary SHMART components as required and is directly controlled by the IOT Processor to transport bio-fluids, diagnostic supplies, cleaning and drying agents between SHMART components—Macerator to Specimen Cartridges to Diagnostic Equipment and Cleaning apparatus.

8. The system as claimed in claim 1, wherein Specimen Cartridges are specially designed for holding in-process BioFluids in preparation of diagnostics and vary by the deployment type of the SHMART system as they hold the specimen in a matrix of predefined consistency so as to allow selected, multiple and repeat diagnostic tests and thus allowing flexibility to configure and revise the diagnostic plan on an as needed basis.

9. The system as claimed in claim 1, wherein Diagnosis Equipment deployed therein varies through a wide range depending on the deployment purpose, level and sophistication of targeted services, and the standard requirement is that the diagnostic equipment must allow automated deployment and includes the features comprising selected from Electronic reporting that directly reads by the IOT Processor; ability to be electronically configured and setup; ability to automate the feed of supplies and allow fitment of sensors that electronically update status of supplies.

10. The system as claimed in claim 1, wherein Supply Cartridges hold the supplies required by the diagnostic equipment and are designed to and automatically feed the supplies based on instructions from the computer.

11. The system as claimed in claim 1, wherein the User Interface that allows the user to interact with the System in a number of ways from a group selected from selecting services required; obtaining a spot report; updating user identity info; viewing disclaimers or other legal information; and authorizing data usage, in addition to using for system maintenance and the mode of communication vary widely from simple LED indicators, to visual electronic display, electronic communication with consumer devices with protocols such as Bluetooth and NFC, depending on the deployment type including printing facility to provide a hardcopy of the interaction.

12. The system as claimed in claim 1, wherein the local database is an electronic store that holds the data in preparation to be uploaded to the Cloud Data Base and it compiles all readings from Sensors, Diagnostic Equipment, Usage Data, User Data, System status, input data and reports of the Onboard Analytics and depending on the deployment type it also contain downloaded information from the Centralized Analytics necessary for User Interface as part of the Remediation Tracking and the local DB is software hosted on the same board as the IOT-Processor or is implemented as separate but attached hardware and in any case the data is secured and encrypted.

13. The system as claimed in claim 1, wherein Onboard Analytics is a set of rules forming the 1st level of intelligence of the system and it considers all the predefined priorities, known patterns and threshold of facts and the data in the local DB to infer other foretelling facts and any corresponding action items that is useful to the users, their medical providers and the Onboard Analytics is usually implemented as software with the IOT-Processor but like the Local DB is on attached hardware.

14. The system as claimed in claim 1, wherein IOT-Network is capable of helping the individual system to communicate with rest of the network especially the Cloud DB in addition to helping the User Interface to communicate with consumer devices with protocols selected from Bluetooth, NFC and IP based Networks and also capable of communicating over long ranges with low power antenna so as to conserve energy and allow long-term unattended operation of the system.

15. The system as claimed in claim 1, wherein BlockChain is a data security mechanism implemented as software-infrastructure that serves to compile and keep track of set of private health data facts collected by the SHMART system at each interaction of each user and allows the users to take ownership of such data and selectively authorize its distribution based upon the User's personal requirements.

16. The system as claimed in claim 1, wherein Bio-Info Wallet is a software folder that contains a key to all the diagnostic information associated with a particular user and the references to the several datasets of that particular user is contained in a bio-info wallet, whereas the actual diagnostic data stored is in the Cloud DB and the information is only transferred between Bio-Info Wallets.

17. The system as claimed in claim 1, wherein Cloud DB is an Information Technology infrastructure that allows massive amounts of data to be hosted with centralized control, yet being rapidly accessible from any part of the network and it provides a cost-effective, secure, scalable and flexible infrastructure to the users and SHMART system administration and consumer organizations.

18. The system as claimed in claim 1, wherein Centralized Analytics is capable of accessing all the sensors, diagnostics and user information along with a set of rules to develop alerts to understand trends that will be useful for Healthcare and also does it on a global scale and accounts for every SHMART system connected.

19. The system as claimed in claim 1, wherein Federated Analytics is similar to Centralized Analytics but differs on the fact that it is conducted by external partners that collaborate within the SHMART ecosystem and so have access to richer knowledgebase to infer the analysis.

20. A method of monitoring the healthiness of the individual or individuals by the Smart Health Monitoring, Analysis and Remediation Tracking (SHMART) system based on Bio-fluid comprising:
a) stepping in and approaching the facility by the user and pressing the service activation button;
b) starting the analysis of the terms of service once the button is pressed;
c) identifying the person who is subjected to evaluation of the health confidentially that is distinguishable by unique user id, if the user id already exists;
d) creating a unique user id as placeholder user id, if not available;
e) smart id subsystem validates the identification of the user;
f) triggers the Monitoring and Diagnostics subsystem to facilitate collection of the Health Data paired up with the User Identification;
g) secures it in the Health Data Control subsystem that is used by Analytics subsystem; and
h) generating remediation actions that are tracked by the Remediation Tracking subsystem to ensure the individual user and the preventive healthcare system as a whole benefit from it.

21. The method of monitoring the healthiness of the individual (s) by the system as claimed in claim 20, wherein Smart Identification is a method of ensuring the identity of the user whose BioFluids are sampled and diagnosed and tracked in a way to provide transparency and traceability required for healthcare and associated research and provide opportunity for commercialization of the data and intelligence resultant from the process, yet allowing the user to maintain ownership and privacy of their personal confidential data.

22. The method of monitoring the healthiness of the individual (s) by the system as claimed in claim 21, wherein the identification of the user of the facility is captured upfront by cross-verification from several foolproof means selected from Biometric tools and physical sign-in and paired up with the associated BioFluid analysis reports by a unique identifier known as a Bio-Info Wallet that is generated and recorded by a BlockChain mechanism that cannot be manipulated and protected from error due to being authenticated by boundless amount of distributed BlockChain services and that the user is afforded the option of pairing the BioFluid diagnostics with their Bio-Info Wallet retroactively after repeated use of the facility by the user all of which is achieved by a set of components and methods designed into the SHMART system comprising a) Analyze Service Requirement b) Monitoring and Diagnostics c) Upfront ID; d) Biometric ID Match; e) Demographic Match; f) Diagnosis Pattern Match; g) External Data Match; h) Decision if Match Found; i) Creating a Bio-Info Wallet; j) Synchronizing a Bio-Info Wallet; k) GPS Location; l) Date/Time Stamp; m) Unit ID; n) Compile Context Info; o) Updating Context into Bio-Info Wallet; p) Update Diagnosis Data into Bio-Info Wallet; q) Health Data Control; r) Analytics; and s) Remedy Tracking

23. The method of monitoring the healthiness of the individual (s) by the system as claimed in claim 20, wherein Health Data Control is a method of ensuring the user of the facility contributing the Bio-Fluid by allowing maximum possibility to claim the ownership of the Health Data and the SHMART system collaborates with the owner of the data to maximize its utility for preventive healthcare purposes and to allow secure storage and transmission of the Health Data as it flows through various actors as necessary for the health care ecosystem to work towards an efficient preventive healthcare system.

24. The method of monitoring the healthiness of the individual (s) by the system as claimed in claim 23, wherein the use of a Bio-Info Wallet consolidates all the references to data relating to a particular owner of the data and any data tractions are managed through BlockChain mechanism infrastructure that cannot be manipulated and is protected from errors due to being authenticated by boundless amount of distributed BlockChain services and the SHMART system has control over Aggregated Data generated from contiguous monitoring and to manage all data in a global-scale, reliable and high-availability environment and with a technology resistant to being obsolete, all of which is achieved by a set of components and methods designed into the SHMART system comprising a) Local DB; b) Personal Data; c) Spot Report; d) Network; e) BlockChain: f) Bio-Info Wallet; g) Wallet Ownership claim; h) Cloud DB Private Data; i) Aggregate Data; j) Cloud DB Aggregate Data; k) 3rd Party Identity Wallet; l) Wallet Owner Authorization; m) Paid Authorization; and n) Contractual Authorization.

25. The method of monitoring the healthiness of the individual (s) by the system as claimed in claim 20, wherein Monitoring & Diagnostics is a method of maximizing the automation necessary for the capture of meaningful facts from the BioFluid sample obtained by the SHMART system from the facility user and handling biofluids from capture, preparation for and conduction of diagnostics, archiving and ultimately to proper disposal in a safest manner possible in compliance to healthcare standards and regulations and allowing autonomous maintenance of the system so as to minimize costs and downtime of the SHMART system and fastest turnaround time between services.

26. The method of monitoring the healthiness of the individual (s) by the system as claimed in claim 25, wherein the use of Sensors are deployed to capture all relevant and possible data from ambience factors of the local environment to levels of supply materials required for diagnostics and to record Perfunctory information of BioFluids and a series of diagnostic equipment is setup to maximize sensitivity to capture any traces of infection or other diagnostically important factors that aid remediation and preventive healthcare and document the results from the Sensors and Diagnostic securely into the Data Control mechanism, all of which is achieved by a set of components and methods designed into the SHMART system comprising a) configuration; b) Ambient Sensors; c) Perfunctory Sensors; d) Supplies Sensors; e) Diagnostics Equipment; f) Completion of Diagnosis; g) Update Diagnostic Data into Bio-Info Wallet; h) Supply Logistics; i) Equipment Maintenance; j) Vendor communication; k) Cleansing & Preparation; and m) Ready to Service.

27. The method of monitoring the healthiness of the individual (s) by the system as claimed in claim 20, wherein Analytics is the method of reaping maximum benefits of preventive healthcare and societal good from the sum of the monitoring and diagnostics data available to the SHMART system and to benefit the user of the facility while collaborating with all possible entities vested in achieving an efficient preventive healthcare and leveraging of knowledge within such entities to the fullest extent possible and positioning the acquired intelligence in a way to harvest it for current and future benefits.

28. The method of monitoring the healthiness of the individual (s) by the system as claimed in claim 27, wherein the use of Onboard Analysis allows an incentive and immediate gratification to the user of the system to encourage repeated participation in the SHMART system and use of Centralized Analysis to create one formidable repository [Cloud DB] of knowledge for benefit of preventive healthcare and using Federated Analytics and Distributed Analytics to enhance collaboration of development of such preventive healthcare knowledge and contribution to the Centralized Analysis and Cloud DB, all of which is achieved by a set of components and methods designed into the SHMART system comprising a) Local DB; b) Onboard Analysis; c) IOT Network; d) Cloud DB; e) Centralized Analysis; f) Authorized Personal Data; g) Aggregate Data; h) Health Providers; i) Medical Researchers; j) Social Organizations; k) Other Collaborators; and l) Federated & Distributed Analytics.

29. The method of monitoring the healthiness of the individual (s) by the system as claimed in claim 20, wherein Remedy Tracking is a method to ensure the individual user using the SHMART facility as well as the society as a whole benefit from the knowledge and intelligence accumulated by the SHMART system and demonstrate such benefit in the form medical attention received by the individual or progress in the medical field towards preventive healthcare and that is especially evident in averting healthcare calamities in nascent stage or improving triage operations when necessary.

30. The method of monitoring the healthiness of the individual (s) by the system as claimed in claim 29, wherein the use of collaborative communication within the SHMART system facilitates tracking the interaction among the individual users, their corresponding medical providers, commercial and social medical research and service organizations and documenting such communication within the Cloud DB, all of which is achieved by a set of components and methods designed into the SHMART system comprising a) User Interaction; b) Consumer Device Communication; c) Authorized Personal Data; d) Healthcare Provider Communication; e) Centralized Communication; f) Aggregate Data; g) Medical Research Organizations; h) Social Organizations; i) Other Collaborators; and j) Federated & Distributed Analytics.

31. The method of monitoring the healthiness of the individual (s) by the system as claimed in claim 20, wherein Remedy Tracking is a method to ensure the individual user, using the SHMART facility as well as the society as a whole benefit from the knowledge and intelligence accumulated by the SHMART system and demonstrate such benefit in the form medical attention received by the individual or progress in the medical field towards preventive healthcare and that is especially evident in averting healthcare calamities in nascent stage or improving triage operations when necessary.

32. The method of monitoring the healthiness of the individual (s) by the system as claimed in claim 31, wherein the use of collaborative communication within the SHMART system facilitates tracking the interaction among the individual users, their corresponding medical providers, commercial and social medical research and service organizations and documenting such communication within the Cloud DB, all of which is achieved by a set of components and methods designed into the SHMART system comprising of a) User Interaction; b) Consumer Device Communication; d) Authorized Personal Data; e) Healthcare Provider Communication; f) SHMART Centralized Communication; g) Aggregate Data; h) Medical Research Organizations; i) Social Organizations; and j) Other Collaborators
